(19) United States
(12) Patent Application Publication
(10) Pub. No.: US 2006/0241560 A1
(43) Pub. Date: Oct. 26, 2006

(54) Convertible absorbent article with extensible side panels

(76) Inventors: Kuo-Shu Edward Chang, Charlotte, NC (US); Patrick King Yu Tsang, Matlock (GB); Andrew Christopher Wright, Chesterfield (GB)

Correspondence Address: FULBRIGHT & JAWORSKI L.L.P., 80 SOUTH EIGHTH STREET, SUITE 2100, MINNEAPOLIS, MN, 55402, US

(21) Appl. No.: 11/113,114

(22) Filed: Apr. 22, 2005

(51) Int. Cl. A61F 13/15

(52) U.S. Cl. 604/385.31; 604/385.3; 604/396

(57) ABSTRACT

A pant-like, refastenable, disposable absorbent article which defines a front waist region, a back waist region and a pair of opposed side panels which extend outwardly from the side edges of the absorbent chassis in the front or back waist region. Each side panel includes at least one elastic zone providing panel extensibility and at least a pair of inextensible zones adjacent an elastic zone. Fastener components of the side panels are used to connect the front waist region to the rear waist region to define the pant-like article. The side panels may also be defined by a pair of side panel margins which extend outwardly from the side edges of the absorbent chassis in both the front waist region and the back waist region and which are joined at the user's side.

### TECHNICAL FIELD

The present invention relates to disposable absorbent articles which are adapted to contain body exudates. More particularly, the present invention relates to prefastened, refastenable, pant-like disposable absorbent articles.

### BACKGROUND

Absorbent articles, such as diapers, training pants and incontinence garments, desirably provide a close, comfortable fit about the wearer and contain body exudates. It is also desirable that such absorbent articles be capable of being pulled up or down over the hips of the wearer to allow the wearer or caregiver to easily pull the article on and easily remove the article if it has not been soiled. For example, such absorbent articles can assist in the toilet training of children. Moreover, it is desirable that such absorbent articles, after being soiled, can be removed from the wearer in a convenient and clean manner without undesirably soiling the caregiver or surrounding area such as the clothes of the wearer.

Conventional diapers have typically included a front waist portion and a rear waist portion, which are releasably connected about the hips of the wearer during use by conventional fasteners, such as adhesive tape fasteners or hook and loop type fasteners. For example, the conventional fasteners have typically included a pair of fasteners, such as adhesive tape tabs, located near the corners of the diaper in the rear waist region of the diaper and a complimentary fastener, such as a taping panel, located on the outer surface of the outer cover of the diaper in the front waist portion of the diaper. In such a configuration, the diaper can be positioned between the legs of the wearer while the wearer is lying down and the adhesive tape tabs then releasably attached to the taping panel to secure the rear waist portion to the front waist portion of the diaper to secure the diaper about the waist of the wearer. Such conventional diapers are easy to fasten about and remove from the wearer after use without undesirably soiling the caregiver. However, such conventional diapers are not provided in a prefastened pant-like configuration and, thus, are not configured to be pulled up or down over the hips of the wearer

The prior art includes examples of absorbent articles which contain body exudates, are capable of being pulled up or down over the hips of the wearer, and provide ease of cleaning and removal after being soiled. For example, some conventional absorbent articles, such as conventional training pants, have included integral side panels which connect the front waist portion to the rear waist portion of the absorbent article. The side panels have been made stretchable such that the waist opening of the absorbent article can expand to allow the absorbent article to be pulled up or down over the hips of the wearer if desired. Such side panels have also been designed such that they may be released to remove the training pant from the wearer after it has been soiled. In other examples, training pants have included side panels which are adapted to be torn apart to release the training pants from the user. Such side panels are not refastenable and require significantly more force to release as compare to known refastenable connectors, e.g. hook and loop fasteners.

Prior art diapers having elasticized waistbands or flaps at their waist regions and elasticized fastening systems have not been completely satisfactory. For example, some waistband fasteners have been narrow, laterally aligned strips and consequently have not always provided a reliable sealing surface with the wearer's body, resulting in leakage. Moreover, the forces exerted on the fastening system have not been efficiently transferred and distributed laterally across the waist region of the diaper along a wide band. As a result, the fit and containment of conventional absorbent articles have not been completely satisfactory.

Accordingly, despite the attempts to develop improved absorbent articles, there remains a need for absorbent articles which can provide the benefits of both conventional training pants and conventional diapers. There remains a need for absorbent articles which conform to the wearer to effectively contain bodily exudates, which are capable of being pulled up and down over the hips and buttocks of the wearer without opening, which are readily secured about and removed from the wearer in a convenient and clean manner, and which allow easy inspection by the caregiver to assist in determining whether the article is soiled.

### BRIEF SUMMARY

In response to the difficulties and problems discussed above, a new prefastened, refastenable, pant-like disposable absorbent article having a pair of improved side panels has been invented.

The present invention concerns a pant-like, refastenable, disposable absorbent article which defines a front waist region, a back waist region, a crotch region which extends between and connects the waist regions, a longitudinal direction and a lateral direction. The absorbent article includes an absorbent chassis which defines an exterior surface, an interior surface opposite the exterior surface, a pair of laterally opposed side edges and a pair of longitudinally opposed waist edges. In one embodiment of the present invention, the absorbent article also includes a pair of opposed side panels which extend outwardly from the side edges of the absorbent chassis in the back waist region of the absorbent article. Each of the opposed side panels defines a side portion which is permanently attached at one end to the absorbent chassis in the back waist region of the absorbent article. Each side panel includes an elastic zone providing panel extensibility, a pair of inextensible zones and a refastenable fastener component. The fastener components of the side panels are used to connect the front waist region to the rear waist region to define the pant-like article. The side panels of embodiments of the present invention may be joined to the absorbent chassis at either a front waist region or a rear waist region.

In another embodiment of the invention, each side panel is defined by a plurality of elastic zones providing panel extensibility and a plurality of inextensible zones. The plurality of elastic zones and inextensible zones of the side panels may assume a variety of different shapes and sizes.

In another embodiment of the invention, the side panels are each defined by a pair of side panel margins which extend outwardly from the side edges of the absorbent chassis in both the front waist region and the back waist region. The absorbent article includes an absorbent chassis and a pair of side panels, each defined by a pair of front and back margins. The absorbent chassis defines an exterior surface, an interior surface opposite the exterior surface, a pair of laterally opposed side edges and a pair of longitudinally opposed waist edges. The back side margins are permanently attached to the absorbent chassis in the back waist region of the absorbent article. The front side margins are permanently attached to the absorbent chassis in the front waist region of the absorbent article. The side panels further include a fastener component for releasably securing the side margins together to define a waist opening and a pair of leg openings to provide the pant-like refastenable, disposable absorbent article. In one embodiment, the pant-like disposable absorbent articles provide overlapping side seams defined by fastener components on the pair of front and back side margins.

An embodiment of the present invention advantageously provides a pant-like, refastenable disposable absorbent article having side panels with fastener components which are elongated and configured in the longitudinal direction to better accommodate the hips and legs of the wearer and thereby provides improved fit and comfort in use when used in a conventional diaper configuration. The present invention provides pant-like disposable absorbent articles which are capable of being reliably pulled up or down over the hips of the wearer to assist in the toilet training of the wearer similar to conventional training pants. Pant-like absorbent articles of the present invention also facilitate readjustment for proper fit on active walking and standing toddlers. Similar to conventional diapers, the pant-like disposable absorbent articles of the present invention can advantageously be applied to and removed from the wearer similar to conventional diapers after they have been soiled with relative ease and cleanliness. Further, the pant-like disposable absorbent articles of the present invention allow easy inspection by the caregiver to assist in determining whether the article is soiled similar to conventional diapers.

Embodiments of the present invention provide improved security and hip coverage when the article is used in the pant-like configuration, as well as greater flexibility and range of motion when used in a conventional diaper configuration.

### BRIEF DESCRIPTION OF THE DRAWINGS

The present invention will be more fully understood and further advantages will become apparent when reference is made to the following detailed description of the invention and the accompanying drawings wherein like numerals represent like elements, and in which:

FIG. 1 representatively shows a perspective view of an example of a prefastened disposable absorbent article of the present invention;

FIG. 2 representatively shows a perspective view of another example of a prefastened disposable absorbent article of the present invention;

FIG. 3 representatively shows a top plan view of the prefastened disposable absorbent article of FIG. 1 in an unfastened, stretched, laid-flat condition with the surface of the article which contacts the wearer facing the viewer;

FIG. 4 representatively shows a plan view of the prefastened disposable absorbent article of FIG. 2 in an unfastened, stretched, laid-flat condition with the surface of the article which contacts the wearer's clothing facing the viewer;

FIG. 5 is a detailed view of a portion of the diaper of FIGS. 1 and 2 illustrating a relaxed or nonexpanded elastic zone.

FIG. 6 is a top plan view of another embodiment of the diaper of FIG. 1 in an unfastened, stretched, laid-flat condition with the surface of the article which contacts the wearer facing the viewer;

FIG. 7a is a top plan view of another embodiment of the diaper of FIG. 1 in an unfastened, stretched, laid-flat condition with the surface of the article which contacts the wearer facing the viewer;

FIG. 7b is a top plan view of another embodiment of the diaper of FIG. 1 in an unfastened, stretched, laid-flat condition with the surface of the article which contacts the wearer facing the viewer;

FIG. 8 is a top plan view of another embodiment of the diaper of FIG. 1 in an unfastened, stretched, laid-flat condition with the surface of the article which contacts the wearer's clothing facing the viewer;

FIG. 9 is top plan view of another embodiment of the diaper in an unfastened, stretched, laid-flat condition with the surface of the article which contacts the wearer facing the viewer; and

FIG. 10 representatively shows a perspective view of another example of a prefastened disposable absorbent article of the present invention.

### DETAILED DESCRIPTION

The present invention concerns pant-like prefastened, refastenable, disposable absorbent articles which are adapted to be worn adjacent to the body of a wearer to absorb and contain various exudates discharged from the body. The prefastened absorbent articles are configured to closely conform to the body of the wearer to effectively contain body exudates while being capable of being pulled up or down over the hips and buttocks of the wearer. The absorbent articles are also refastenable such that they can be secured to and removed directly from the waist of the wearer and easily inspected to determine if they have been soiled during use. As such, the prefastened, refastenable, disposable absorbent articles of the present invention can function in a similar manner to conventional training pants when left in the prefastened configuration or they can be unfastened prior to or during use to function in a refastenable manner similar to conventional diapers.

The pant-like disposable absorbent articles of the present invention will be described in terms of a disposable, prefastened diaper article which is adapted to be worn about the lower torso of a user. In particular, the prefastened disposable absorbent articles will be described in terms of a prefastened, refastenable, disposable diaper having prefastened side panels and at least a pair of refastenable fasteners. It is understood that the articles and methods of the present invention are equally adaptable for other types of absorbent articles, such as adult incontinent products, training pants, feminine hygiene products, other personal care or health care garments, and the like.

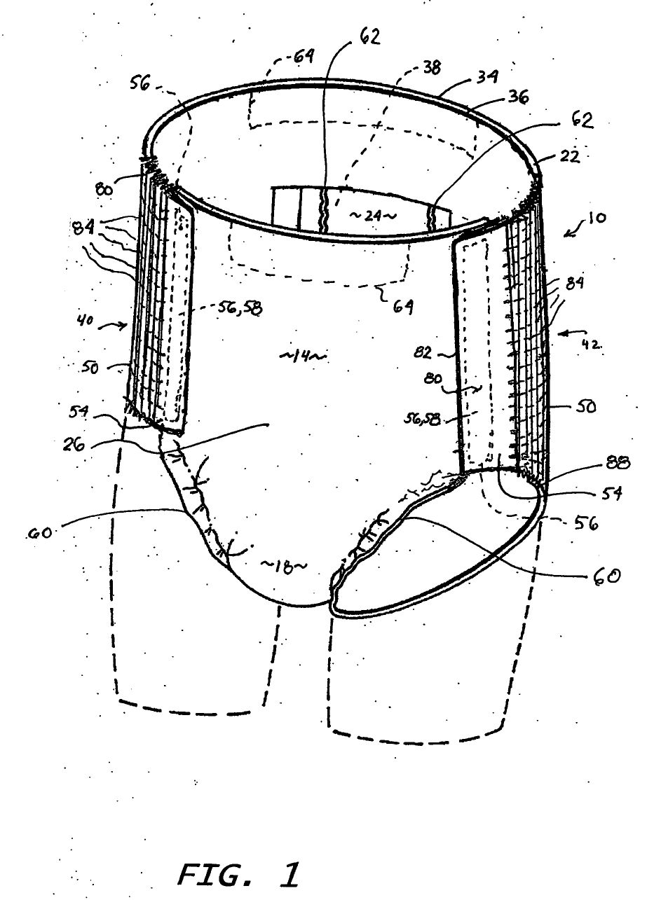

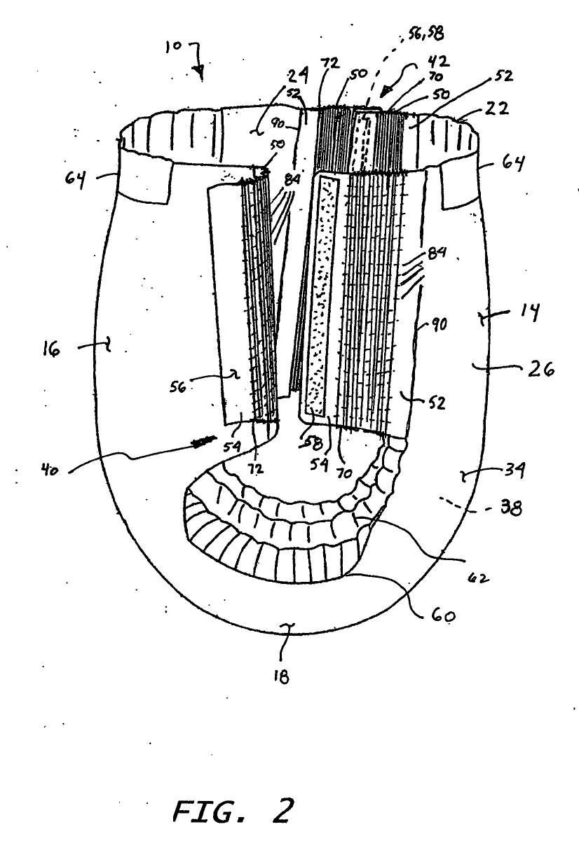

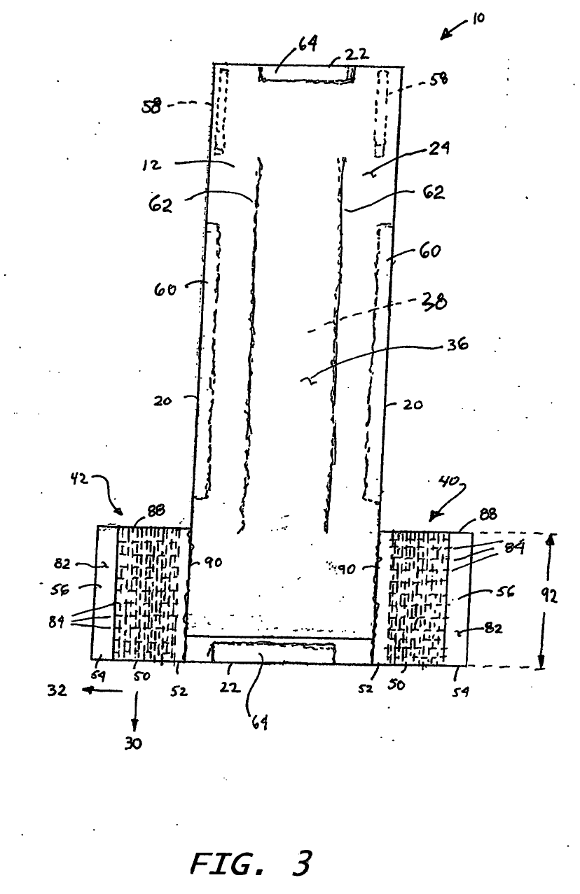

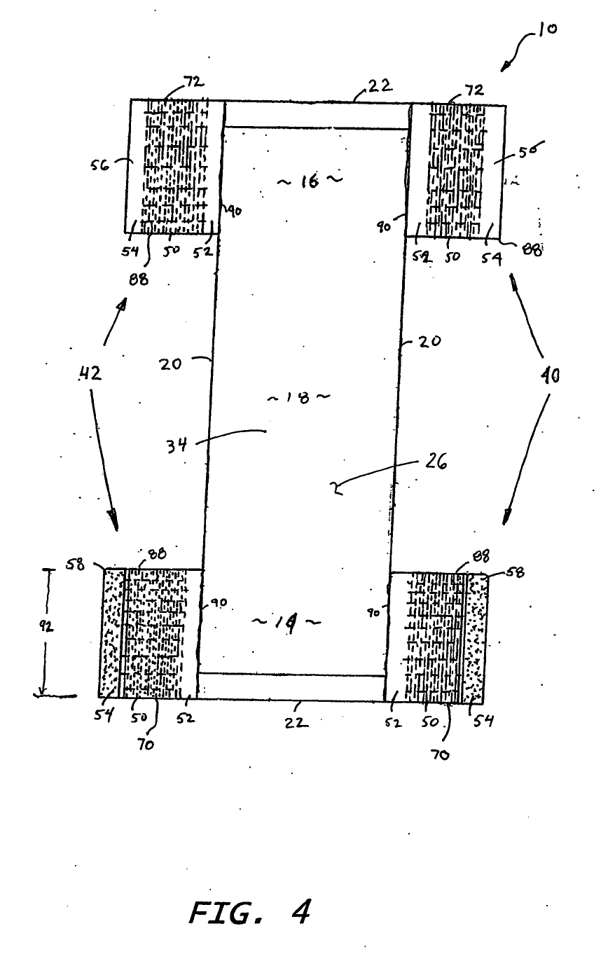

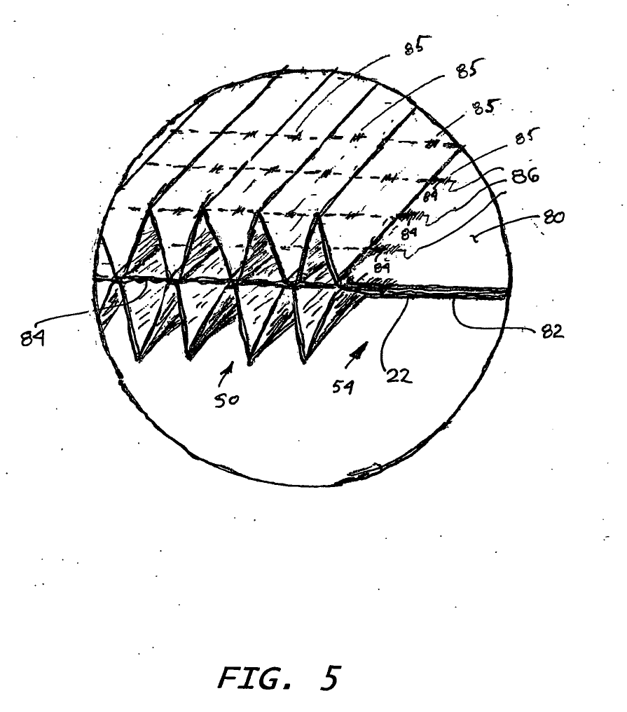

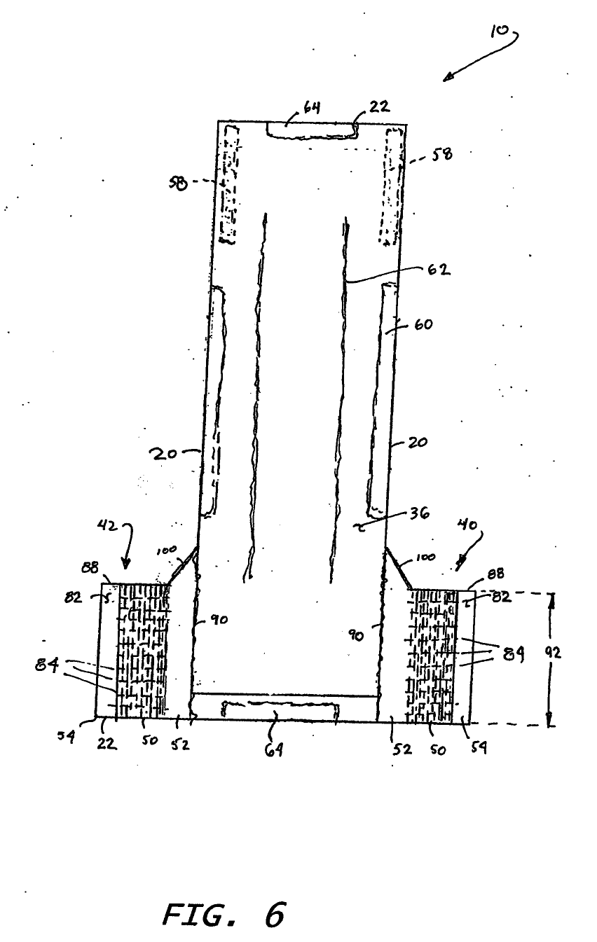

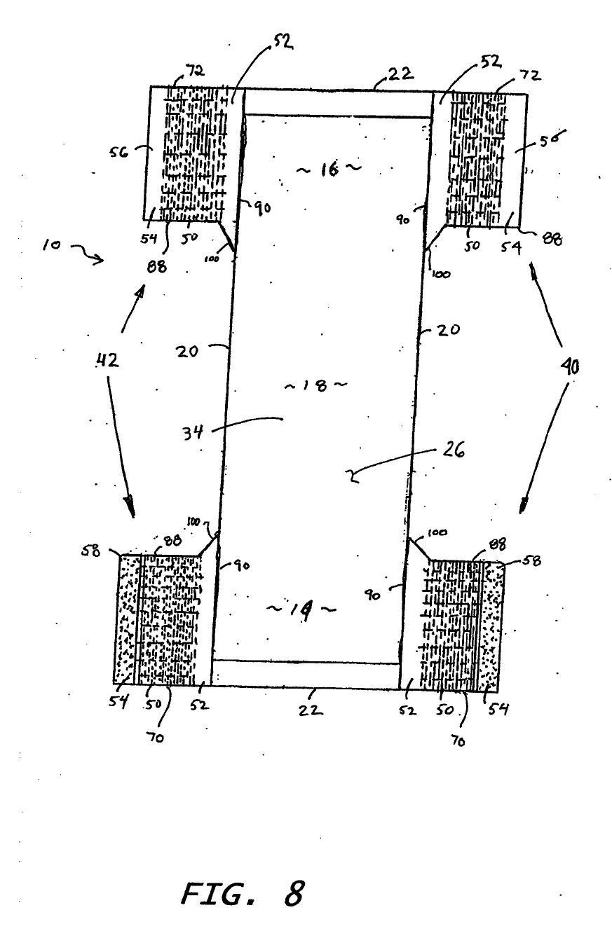

FIG. 1 representatively shows one example of a pant-like prefastened, refastenable disposable diaper, as generally indicated at 10, of the present invention. FIG. 2 representatively illustrates an alternative embodiment of the pant-like prefastened disposable diaper of the present invention. FIG. 3 illustrates the prefastened diaper of FIG. 1 in a stretched and laid flat state and with the portion of the diaper which faces towards the wearer facing the viewer. FIG. 4 illustrates the prefastened diaper of FIG. 2 in a stretched and laid flat state and with the portion of the diaper which faces away from the wearer facing the viewer. FIG. 5 illustrates, in detail, an expanded portion of the diaper 10. FIGS. 6-10 each illustrate a different embodiment of the present invention.

The illustrated diapers 10 include an absorbent chassis 12 (FIG. 3, for example) which generally defines a front waist region 14, a rear waist region 16, and a crotch region 18 which extends between and connects the front and rear waist regions 14 and 16. The absorbent chassis 12 further defines a pair of laterally opposed side edges 20, a pair of longitudinally opposed waist edges 22, an interior body facing surface 24 which is configured to contact the wearer, an outer garment facing surface 26 opposite the interior surface 24 which is configured to contact the wearer's clothing in use, a longitudinal direction 30, and a lateral direction 32 (FIG. 3). The absorbent chassis 12 may include an outer cover 34, a body side liner 36 which is connected to the outer cover 34 in a superposed relation, and an absorbent core 38 which is located between the outer cover 34 and the body side liner 36.

The front waist region 14 comprises the portion of the diaper 10 which, when worn, is positioned on the front of the wearer while the rear waist region 16 comprises the portion of the diaper 10 which, when worn, is positioned on the back of the wearer. The crotch region 18 comprises the portion of the diaper 10 which, when worn, is positioned between the legs of the wearer and covers the lower torso of the wearer. The laterally opposed side edges 20 of the absorbent chassis 12 of the diaper 10 generally define portions of the leg openings. The waist edges 22 of the absorbent chassis 12 of the diaper 10 are configured to encircle the waist of the wearer when worn and provide a waist opening when fastened that defines a waist perimeter dimension.

Diaper 10 also includes extensible side panels 40, 42 which releasably connect the rear waist region 16 to the front waist region 14 to provide the pant-like prefastened absorbent article. Each side panel 40, 42 includes an elastic zone 50 disposed between a pair of inextensible zones 52, 54 and a fastener component 56 preferably disposed within at least a portion of inextensible zone 54 as described in further detail hereinafter. Fastener component 56 engages a corresponding fastener component 58 on the absorbent chassis 12.

In the embodiment illustrated in FIG. 1, the elastic side panels 40, 42 are temporarily secured at the front waist region 14 to form the pant-like article. In alternative embodiments, the elastic side panels 40, 42 may be temporarily secured at the rear waist region 16. In the illustrated embodiment of FIG. 1, the free ends of the elastic side panels 40, 42 are temporarily secured on an outer surface of the front waist region 14. In alternative embodiments, the free ends of the elastic side panels 40, 42 may be temporarily secured to an inner surface of the front waist region 14 or rear waist region 16. Desirably, diaper 10 includes refastenable fastener components 56, 58 which provide inspection access to the diaper 10 interior and the ability to vary the size of the waist opening over a wide range to fit the waist of the wearer.

Before the diaper 10 is placed on the wearer, the fastener components 56, 58 may be secured to provide a prefastened, refastenable disposable diaper, such as provided by example in FIGS. 1, 2 and 10. In such a configuration, the prefastened diaper 10 can be pulled on or off over the legs and hips of the wearer. If the prefastened diaper 10 becomes soiled during use, the fastener components 56, 58 can be disengaged from each other to easily remove the diaper 10 from the waist of the wearer with reduced risk of undesirably soiling the clothes or legs of the wearer. The side panels 40, 42 can also be easily disengaged to inspect the diaper 10 for possible soiling or to first apply the product to the wearer if desired. Thus, the diaper 10 is configured to be pulled on or off over the hips of the wearer such as conventional training pants and can be readily applied or removed by disengaging the fasteners similar to conventional diaper articles. Moreover, the fastener components 56, 58 can be repositioned if necessary after the prefastened diaper 10 has been pulled on over the legs and hips of the wearer to adjust the fit of the diaper to the wearer.

The diaper 10 may further include leg elastics 60, containment flaps 62, and waist elastics 64 as are known to those skilled in the art. The absorbent chassis 12 of the disposable diaper 10 may include a pair of containment flaps 62 which are configured to provide a barrier to the lateral flow of body exudates. The containment flaps 62 may be located along the laterally opposed side edges 20 of the absorbent chassis 12. Each containment flap 62 typically defines an unattached edge which is configured to maintain an upright, perpendicular configuration in at least the crotch region 18 of the diaper 10 to form a seal against the wearer's body. The containment flaps 62 may extend longitudinally along the entire length of the absorbent chassis 12 or may only extend partially along the length of the absorbent chassis 12. Such containment flaps 62 are generally well known to those skilled in the art.

The disposable diaper 10 may further optionally include elastics at the waist edge 22 of the absorbent chassis 12 of the diaper 10 to further prevent leakage of body exudates and support the absorbent chassis 12. For example, the disposable diaper may further comprise elastic waist features 64 that help provide improved fit and containment of body exudates.

The diaper 10 of the present invention may also include a pair of leg elastic members 60 which are connected to the laterally opposed side edges 20 of the absorbent chassis 12 in the crotch region 18 of the diaper 10. The leg elastics 60 are generally adapted to fit about the legs of a wearer in use to maintain a positive, contacting relationship with the wearer to effectively reduce or eliminate the leakage of body exudates from the diaper 10. Materials suitable for use as the leg elastics 60 and waist elastic 64 are well known to those skilled in the art. Exemplary of such materials are sheets or strands or ribbons of a polymeric, elastomeric material which are adhered to the outer cover 34 in a stretched position, or which are attached to the outer cover 34 while the outer cover is pleated, such that elastic constrictive forces are imparted to the outer cover 34. The leg elastics 60 may also include such materials as polyurethane, synthetic and natural rubber. Leg elastics 60, containment flaps 62 and waist elastics 64 may include elastic foam materials, elastic films (apertured, woven and non-woven, for example), elastic scrim material, elastic non-woven materials, elastic composites, and selectively activated elastic materials.

The diaper 10 may be of various suitable shapes. For example, in the unfastened configuration, the diaper may have an overall rectangular shape, T-shape or an approximately hourglass shape. In the illustrated embodiments, the diaper has an overall rectangular shape in an unfastened and flattened configuration. The various aspects and configurations of the invention can provide distinctive combinations of softness, body conformity, reduced red-marking of the wearer's skin, reduced skin hydration, improved containment of body exudates and improved aesthetics.

The various components of the diaper 10 may be integrally assembled together employing various types of suitable attachment means, such as adhesive, sonic and thermal bonds or combinations thereof. In the illustrated shown embodiments, for example, the outer cover 34 and body side liner 36 are joined to each other. Similarly, other diaper components, such as the leg elastics 60 and the primary fasteners 62, may be assembled into the diaper 10 by employing the above-identified attachment mechanisms. Desirably, the majority of the diaper components are assembled together using ultrasonic bonding techniques for reduced manufacturing cost and improved performance.

The outer cover 34 of the absorbent chassis 12 of the diaper 10 may suitably be composed of a material which is either liquid permeable or liquid impermeable. It is generally preferred that the outer cover 34 be formed from a material that is substantially impermeable to liquids. A typical outer cover can be manufactured from a thin plastic film or other flexible liquid-impermeable material. For example, the outer cover 34 may be formed from a polyethylene film. If it is desired to present the outer cover 34 with a more cloth-like feeling, the outer cover 34 may comprise a polyolefin film having a nonwoven web laminated to the outer surface thereof, such as a spunbond web of polyolefin fibers. For example, a stretch-thinned polypropylene film may have thermally laminated thereto a spunbond web of polypropylene fibers. Methods of forming such cloth-like outer covers are known to those skilled in the art.

Further, the outer cover 34 may be formed of a woven or nonwoven fibrous web layer which has been totally or partially constructed or treated to impart a desired level of liquid impermeability to selected regions that are adjacent or proximate the absorbent core 38. Still further, the outer cover 34 may optionally be composed of a micro-porous “breathable” material which permits vapors to escape from the absorbent core 38 while still preventing liquid exudates from passing through the outer cover 34. For example, the outer cover 34 may comprise a stretched microporous polyolefin film having a nonwoven web laminated to the outer surface thereof, such as a spunbond web of polyolefin fibers. The outer cover 34 can also be embossed or otherwise provided with a matte finish to provide a more aesthetically pleasing appearance.

The bodyside liner 36 suitably presents a bodyfacing surface which is compliant, soft feeling, and nonirritating to the wearer's skin. Further, the bodyside liner 36 may be less hydrophilic than the absorbent core 38, to present a relatively dry surface to the wearer, and may be sufficiently porous to be liquid permeable, permitting liquid to readily penetrate through its thickness. A suitable bodyside liner 36 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 36 is suitably employed to help isolate the wearer's skin from liquids held in the absorbent core 38.

Various woven and nonwoven fabrics can be used for the bodyside liner 36. For example, the bodyside liner may be composed of a meltblown or spunbonded web of polyolefin fibers. The bodyside liner may also be a bonded-carded web composed of natural and/or synthetic fibers. The bodyside liner may be composed of a substantially hydrophobic material, and the hydrophobic material may, optionally, be treated with a surfactant or otherwise processed to impart a desired level of wettability and hydrophilicity. The bodyside liner 36 may further include a lotion or treatment applied thereto to which is configured to treat or be transferred to the wearer's skin.

The absorbent core 38 of the diaper 10, may suitably comprise a matrix of hydrophilic fibers, such as a web of cellulosic fluff, mixed with particles of a high-absorbency material commonly known as superabsorbent material. In a particular embodiment, the absorbent core 38 comprises a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent core 38 to better contain and absorb body exudates. The concentration of the superabsorbent particles may also vary through the thickness of the absorbent core 38. Alternatively, the absorbent core 38 may comprise a laminate of fibrous webs and superabsorbent material or other suitable means of maintaining a superabsorbent material in a localized area.

The high-absorbency material can be selected from natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful in the present invention. Such high-absorbency materials are well known to those skilled in the art and are widely commercially available.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent body in an amount of from about 5 to about 100 weight percent based on total weight of the absorbent core 38.

The absorbent core 38 may have any of a number of shapes. For example, the absorbent core may be rectangular, I-shaped, or T-shaped. In the embodiments of FIGS. 1-6, the absorbent core is generally rectangular in shape. The size and the absorbent capacity of the absorbent core 38 should be compatible with the size of the intended wearer and the liquid loading imparted by the intended use of the absorbent article.

Side panels 40, 42 are used to selectively and refastenably connect the rear waist region 16 to the front waist region 14 to form a pant-like wearable diaper. According to one embodiment of the present invention, each side panel 40, 42 includes an elastic zone 50, a pair of inextensible zones 52, 54 and a fastener component 56. FIGS. 1-4 illustrate diapers 10 having two different versions of side panels 40, 42. In the embodiment of FIG. 1 and FIG. 3, the side panels 40, 42 include a fastener component 56 which is refastenably engaged with another fastener component 58 at the outer garment facing surface 26 of the front waist region 14 of the diaper 10. In a related embodiment, the side panels 40, 42 may refastenably engage another fastener component at the surface 26 of the rear waist region 16 of the diaper, i.e, the side panels 40, 42 are temporarily secured and disengaged at the rear waist region 14. In the embodiment of FIG. 2 and FIG. 4, the side panels 40, 42 include a pair of side panel margins 70, 72 which are refastenably secured together via fastener components 56, 58. Side margin 70 is permanently joined near the side edge 20 of the absorbent chassis 12 in the front waist region 14 and side margin 72 is permanently joined near side edge 20 of the absorbent chassis 12 in the rear waist region 16. The side margins 70, 72 are releasably and refastenably attached together via fastener components 56, 58 to form the pant-like article.

Elastic zone 50 of the side panels 40, 42 provides for panel extensibility and may be provided in a variety of ways. For example, elastic zone 50 may be a single piece of stretchable elastic material or a combination of individual pieces or panels of elastic material. A variety of alternative structures would be appreciated by those skilled in the art. The elastic zone 50 or at the least individual pieces or panels of the elastic zone 50 may comprise elastic composite materials or non-stretchable materials which have been rendered elastically contractible by means known to those skilled in the art.

In the illustrated embodiments of diaper 10, elastic zone 50 includes a two-ply stretch panel comprising a top layer 80, a bottom layer 82 and a plurality of intermediate elastic ribbons 84. Ribbons 84 are illustrated in particular detail in FIG. 5. Elastic ribbons 84 are generally laterally aligned and include ends 86 which are secured near lateral boundaries of the elastic zone 50. Elastic Ribbons 84 may be secured at or near ends 86 to top layer 80, bottom layer 82 or both. In the illustrated embodiment, elastic ribbons 84 are intermittently secured to the top layer 80 and the bottom layer 84 as indicated by numeral 85. In another embodiment (not shown), the elastic ribbons 84 may be secured only at theirs ends and intermediate portions of the ribbons 84 may be generally unrestrained and movably engage portions of the stretch panel as the stretch panel is biased during diaper 10 application, use or removal. Elastic ribbons 84 are preferably pretensioned prior to being attached between layers 80, 82. Each elastic ribbon 84 may include multiple elastic fibers or may include a single elastic fiber. In the illustrated embodiments of the diaper 10, the elastic zone 50 extends in the longitudinal direction 30 from the waist edge 22 to a leg opening edge 88, and as a result, generally the entire longitudinal width of the side panel 40, 42 is elastically deformable within the elastic zone 50.

In another alternative configuration, elastic zone 50 may be provided by a substantially non-elastomeric material, such as polymer films, woven fabrics, non-woven fabrics, or the like such as described above as being suitable for the outer cover 34 or bodyside liner 36. Such a elastic zone 50 could be modified to render portions elastically contractible to provide the desired elastic properties. For example, the elastic zone 50 may be made elastic or stretchable by means well known to those skilled in the art. For example, different portions of the elastic zone 50 may include a plurality of elastic strands which are elongated and adhesively attached in the lateral direction 32 to the respective portions of the elastic zone. Suitable elastic strands are known to those skilled in the art. Alternatively, a piece of elastic material may be adhesively attached to the respective portions of the elastic zone 50 in an elongated condition to provide the desired stretchability. In one embodiment, the piece of elastic material may be applied to the stretch panel in a pretensioned configuration. In another example, the elastic material may be latent, in which case the elastic material would initially have non-elastomeric properties, but would later be activated to impart elastomeric properties to the stretch panel by any of various means known to those skilled in the art. Suitable latent elastic materials are well known to those skilled in the art.

In the diaper 10 embodiments of FIGS. 1-4, side panels 40, 42 and side margins 70, 72 also include a pair of inextensible zones 52, 54. The inextensible zones 52, 54 may be provided in a variety of ways. For example, the inextensible zones 52, 54 may each be a single ply of material or a combination of individual panels or plies of material. In another diaper embodiment, the inextensible zones 52, 54 may be formed from materials of outer cover 34 and/or bodyside liner 36. In the illustrated embodiment of diaper 10, the inextensible zones 52, 54 are defined by a two-ply panel comprising a top layer 80 and a bottom layer 82. As used herein, the term “inextensible” refers to a material property of being substantially less elastic or stretchable as compared to material properties of the elastic zone 50. Depending upon the material selected to define the inextensible zones 52, 54, such zones may have slight elastic or stretchable properties.

Side panels 40, 42 and side margins 70, 72 are joined at boundaries of inextensible zone 52 to the absorbent chassis 12 along attachment lines 90. A variety of joining means are known in the art including adhesives, thermal bonds, etc. In illustrated embodiments of the diaper 10, the inextensible zones 52, 54 extend in the longitudinal direction 30 from the waist edge 22 to a leg opening edge 88. In other embodiments of the diaper 10, the inextensible zones 52, 54 may extend in the longitudinal direction 30 from near the waist edge 22 to near a leg opening edge 88. In a preferred embodiment of the present invention, side panels 40, 42 may be joined to absorbent chassis 12 at either the front waist region 14 or the rear waist region 16.

In the embodiments of FIGS. 1-4, the longitudinal length 92 of the side panel inextensible zones 52, 54 is substantially equal to the longitudinal length of the elastic zone 50. In the embodiments of FIGS. 6-8, the longitudinal length of the inextensible zone 52 is generally greater than the longitudinal length 92 of the elastic zone 50 and the other inextensible zone 54. In the illustrated embodiments of FIGS. 6-8, the inner inextensible zone 52 is defined by an outwardly narrowing edge 100. The benefits of this feature include better fit, better body coverage, and better overall aesthetics and pant-like appearance.

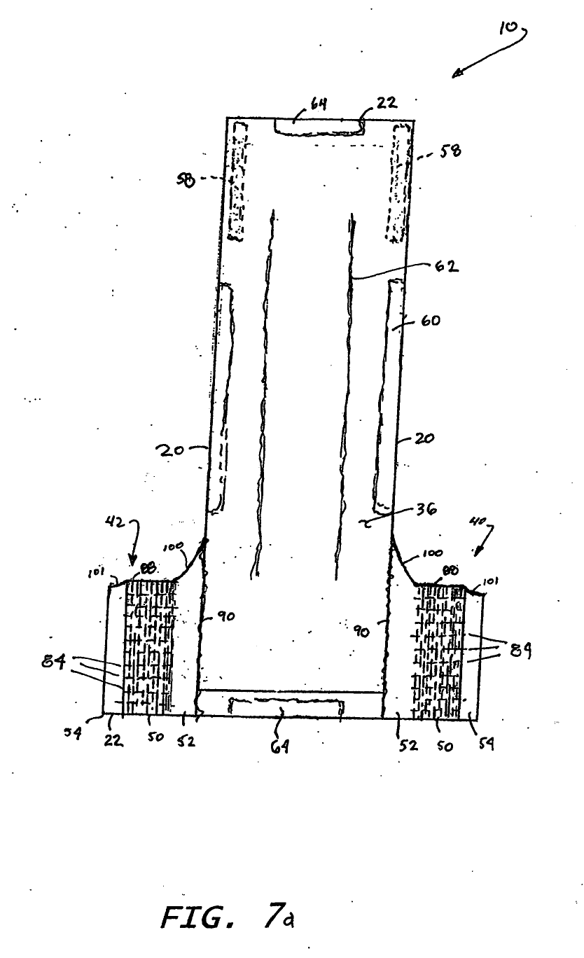

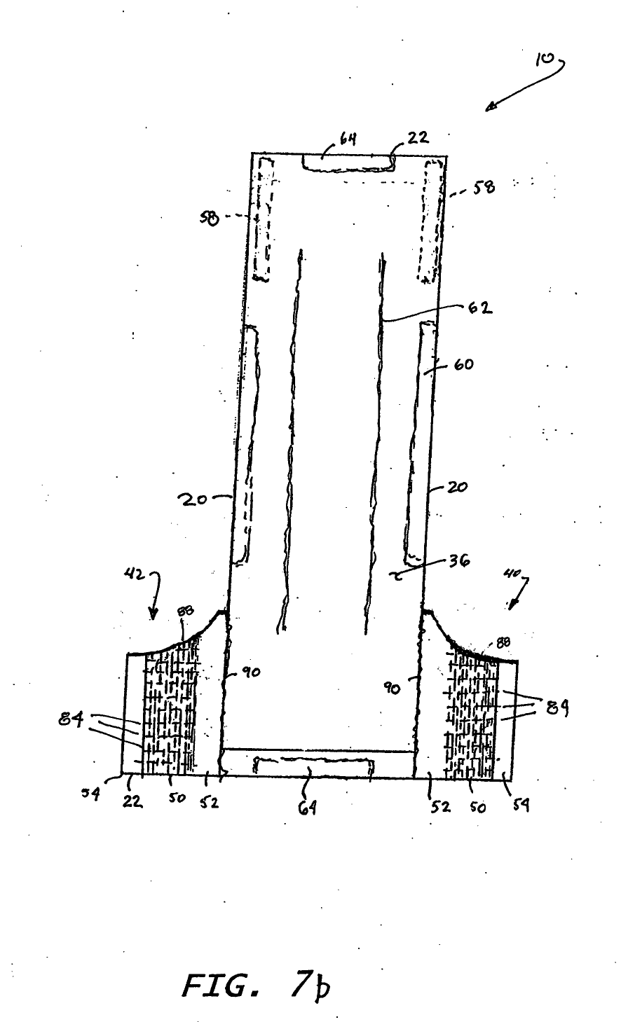

In the embodiment of FIG. 7a, the longitudinal length of the inextensible zone 52 is generally greater than the longitudinal length 92 of the elastic zone 50 and the other inextensible zone 54. The inner inextensible zone 52 is defined by an outwardly narrowing edge 100. Edge 100 is generally concavely configured between side edge 20 of the absorbent chassis 12 and leg edge 88. The outer inextensible zone 54 is also defined by an outwardly narrowing edge 101. Edges 88, 100 may assume a variety of shapes including linear or nonlinear configurations. As a result, that portion of the leg opening defined by side panels 40, 42 may assume a variety of different configurations. For example, the inextensible zones 52, 54 may define a concavely shaped leg opening edge and the elastic zone 50 may define a linear leg opening edge which is generally parallel to the waist edge 22.

In the embodiment of FIG. 7b, the longitudinal length of the inextensible zone 52 is greater than the longitudinal length 92 of the elastic zone 50 and the other inextensible zone 54. In this embodiment, the leg edge 88 of inextensible zones 52, 54 and elastic zones includes a narrowing curved edge. Leg edge 88 is generally concavely curved across a portion of inextensible zone 52, elastic zone 50 and outer inextensible zone 54. Leg edge 88 may assume a variety of shapes including linear or nonlinear configurations.

Fastener component 56 is preferably disposed entirely within inextensible zone 54. The fastener component 56 is one part of a fastening system which may also include fastener components 58 located on front waist region 14 of the diaper 10 (FIGS. 1, 3, 6, 7, 9, 10) or within inextensible zone 54 of a side margin 70 (FIGS. 2, 4, 8). The provision of refastenable fasteners 56, 58 allows for ease of securing and removing the diaper 10 from the waist of the wearer without undesirably soiling the wearer. The provision of refastenable fasteners 56, 58 further readily allows for the inspection of the inside of the diaper 10 to determine if it has been soiled with the ability to refasten if it is not soiled.

Suitable fasteners 56, 58 are well known to those skilled in the art and can include adhesive tape tab fasteners, hook and loop fasteners, coadhesive fastening systems, mushroom fasteners, snaps, pins, belts and the like, and combinations thereof. For example, as representatively illustrated in FIG. 1, the fastening system may include hook type fasteners 58 joined to the absorbent chassis 12 at the front waist portion 14 and a complimentary loop type fastener 56 defined on the side panels 40, 42 within inextensible zone 54. The fastening system may include hook type fasteners 58 joined to the absorbent chassis 12 at the front waist region 14 or the rear waist region 16 and complimentary loop type fasteners 56 defined on the side panels 40, 42. In other embodiments, the loop type fasteners 56 may be provided at the front or rear waist regions 14, 16 of the absorbent chassis 12 with complementary hook type fasteners 58 provided on the side panels 40, 42. In another example, the fastening system may include hook type fasteners 58 joined on a side margin 70 of the side panels 40, 42 and a complimentary loop type fastener 56 defined on the other side margin 72. Desirably, the fastener components 56, 58 are hook and loop type fasteners which are refastenably engageable as such an arrangement provides the ability to vary the size of the waist opening in very small increments over a wide range to fit the waist of the wearer.

In the embodiments of diaper 10 illustrated in FIGS. 1-4 and 6-10, the fastener system includes elongated hook and loop fasteners 56, 58 which are aligned generally in the longitudinal direction 30 of diaper 10. The side panel fastener component 56 extends generally between the waist edge 22 and the leg opening edge 88. As a result, fastening forces are desirably distributed across a wide area of the outer inextensible zone 54.

The longitudinal length 92 of the side panels 40, 42 and side margins 70, 72 is generally sufficient to effectively distribute the fastening forces across a wide area of the absorbent chassis 12 to provide improved fit without excessive irritation of the skin of the wearer. Desirably, the side panels defines a longitudinal length 92 which is desirably from about 5 percent to about 35 percent of the overall longitudinal length of the diaper 10. A more preferred range of lengths 92 is from about 10 percent to about 25 percent of the overall length of the diaper 10. Lengths less than the above-specified ranges may not provide the improved fit and appearance of the article on the wearer and may result in excessive red marking. Whereas, lengths greater than the above specified ranges may extend too far into the crotch region 18 of the article such that the side panel may interfere with the deposition of body exudates into the diaper article.

The fastener system, including fastener components 56, 58, may comprise attachment means known in the art including pressure sensitive adhesives, co-adhesive materials, mechanical fasteners such as hook and loop type fasteners, or any combination of these or any other attachment means known in the art. Exemplary adhesive tape tab fastening systems are disclosed in U.S. Pat. No. 3,848,594 entitled “Tape Fastening System for Disposable Diaper” issued to Buell on Nov. 19, 1974; and U.S. Pat. No. 4,662,875 entitled “Absorbent Article” issued to Hirotsu and Robertson on May 5, 1987. Exemplary fastening systems comprising mechanical fastening components are described in U.S. Pat. No. 5,064,167 entitled “Mechanical Fastening Prong” issued to Thomas Oct. 14, 1991; U.S. Pat. No. 4,869,716 entitled “Mechanical Fastening Systems With Adhesive Tape Disposal Means For Disposal of Absorbent Articles” issued to Scripps on Sep. 18, 1989; and U.S. Pat. No. 4,838,815 entitled “Disposable Diaper Having an Improved Fastening Device” issued to Scripps on Jul. 11, 1989. An example of a fastening system having combination mechanical/adhesive fasteners is described in U.S. Pat. No. 4,938,527 entitled “Pressure-Sensitive Adhesive Fastener and Method of Making Same” issued to Battrell on Aug. 7, 1990. Each of these patents are incorporated herein by reference.

In a preferred embodiment of the present invention, the fastener components 56, 58 comprise hook and loop type fasteners. As used herein, the term “hook and loop type fasteners” refers to fastening means comprising a “hook” component (hereinafter referred to as an “engaging component”) and a complementary loop component (hereinafter referred to as a “landing component”). The term “hook” is used to designate a material having engaging elements. Thus, the hook fastening material may also be referred to as a male fastener. It should also be understood that the use of the term “hook” should be non-limiting in the sense that the engaging elements may comprise any shapes as are known in the art so long as they are adapted to engage a complementary landing component. In the illustrated embodiments, fastener component 58 defines the hook component and fastener component 56 defines the landing component.

Thus, the hook component may be manufactured from a wide range of materials. Further, the engaging component may have any shape such as hooks, “T's”, “mushrooms” or any other shape as are well known in the art. Suitable materials include nylon, polyester, polypropylene, or any combination of these materials.

The hook components may be joined to the absorbent article 10 by any suitable means. They may be directly joined to the outer surface of the absorbent chassis 12, or mounted on some intermediate member, such as a tape tab or other tab member. The tab or separate material may be of any shape or size and may comprise any suitable materials, including single materials or laminates. Further, the tab or separate material may have stretch properties if desirable. Examples of materials which could be used as an intermediate tab include films, wovens, and nonwovens, and the like, or any combination thereof. The tab or the separate material may be joined to the absorbent chassis 12 by any means known in the art, including, but not limited to heat, pressure, ultrasound, adhesive, cohesive or any combination of these or other bonding means.

The landing component preferably comprises a fastening element engageable with the hook component. Thus, the landing component may be manufactured from a wide range of materials and configurations capable of securely engaging the engaging component.

In one preferred embodiment, the landing component is fastening component 56 and comprises a plurality of fiber elements, such as a loop fastening material, that engage the engaging elements of the engaging component. The loop fastening material may be manufactured from a wide range of materials to provide fiber elements, preferably loops. Suitable materials include woven materials, nonwovens, nylons, polyesters, polypropylenes, or any other known loop fastening materials or combination of these materials. The landing component may be integrated into other components of the diaper 10 or may be a separate component which is joined with the diaper 10 by any means known in the art, including but not limited to adhesives, coadhesives, heat, pressure, ultrasound, or any combination thereof. In one preferred embodiment of diaper 10, the landing component is provided by the surface of the outer cover 34 within front waist region 14 (FIG. 1, for example). In another preferred embodiment of diaper 10, the landing component is defined by a material surface within inextensible zone 54 of side margin 72. In yet another embodiment, the landing component may be provided by the surface of the outer cover 34 within the rear waist region 16. Fastening component 56 (the landing component of illustrated embodiments) may either be a discrete separate element or a unitary element which is neither divided nor discontinuous with another element of the diaper 10, such as the outer cover 34 or bodyside liner 36. It should be noted that the hook components and the landing components may assume a shape and size different from the illustrated embodiments. Further, embodiments are contemplated wherein the hook components, the landing components or both are not continuous, but rather include a number of individual members that provide the requisite fastening characteristics and to allow for the best fit for a broad range of wearers.

It is also generally desirable that the side panels 40, 42 and side margins 70, 72 are formed from materials which are substantially impermeable to liquids and which provide a more clothlike feeling. Such materials are capable of maintaining a close conforming fit while not irritating the skin of the wearer. The side panels 40, 42 and side margins 70, 72 can be provided in any suitable manner which provides the desired fit properties and performance. For example, the side panels 40, 42 and side margins 70, 72 may be provided by multiple individual pieces of material, some of which may be elastomeric, which are joined together along their side edges to provide the side panel 40, 42 and side margins 70, 72.

The side panel 40, 42 and side margins 70, 72 of the different embodiments of the present invention may be attached to the absorbent chassis 12 of the diaper 10 in any suitable manner which provides the desired properties. For example, the side panel may be attached to the absorbent chassis 12 using adhesive, ultrasonic, thermal bonding techniques and the like or combinations thereof. As representatively illustrated in the drawings, side panels 40, 42 and side margins 70, 72 are attached to the absorbent chassis of the diaper 10 along attachment lines 90. In the illustrated embodiments, attachment lines 90 are generally linear and aligned in the longitudinal direction 30. Alternative non-linear and/or angled attachment zones may also be practicable.

In alternative embodiments of the present invention, the side panels 40, 42 and side margins 70, 72 may be integrated with components of the absorbent chassis of the diaper. For example, the side panels 40, 42 and side margins 70, 72 may comprise lateral extensions of the outer cover 34 and bodyside liner 36. Further, the side panels 40, 42 and side margins 70, 72 of the present invention may be joined to the absorbent chassis 12 of the diaper 10 in any location which provides the desired properties. As used herein, the term “joined” encompasses configurations whereby an element is directly secured to the other element by affixing the element directly to the other element, and configuration whereby the element is indirectly secured to the other element by affixing the element to intermediate member(s) which in turn are affixed to the other element.

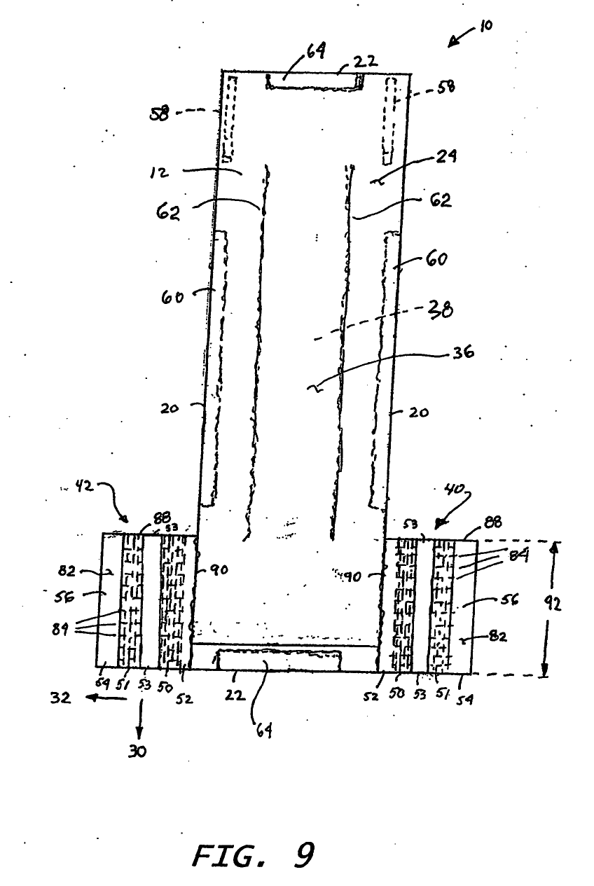

Side panels 40, 42 and side margins 70, 72 may assume different sizes, shapes, configurations and materials. The exact length, width and thickness of the side panels 40, 42 and side margins 70, 72 may vary depending on the intended application. For example, FIG. 9 illustrates yet another embodiment of the present invention wherein side panels 40, 42 each comprise a plurality of elastic zones 50, 51 and inextensible zones 52, 53, 54. As illustrated in FIG. 9, absorbent articles according to the present invention may have m elastic zones and m+1 inextensible zones, where m is 2 or greater. The zones 50-54 may assume a variety of different shapes and sizes. As illustrated, zones 50-54 have the same longitudinal length 92. In other embodiments, the zones 50-54 may have a different lengths, widths or thicknesses. Side panels 40, 42 of FIG. 9 may include a plurality of separate panels which are joined in the manufacturing process. In other embodiments of the present invention, the side panels 40, 42 may emanate near the front waist region 14 and be temporarily affixed at the rear waist region 16 to define a pant-like article.

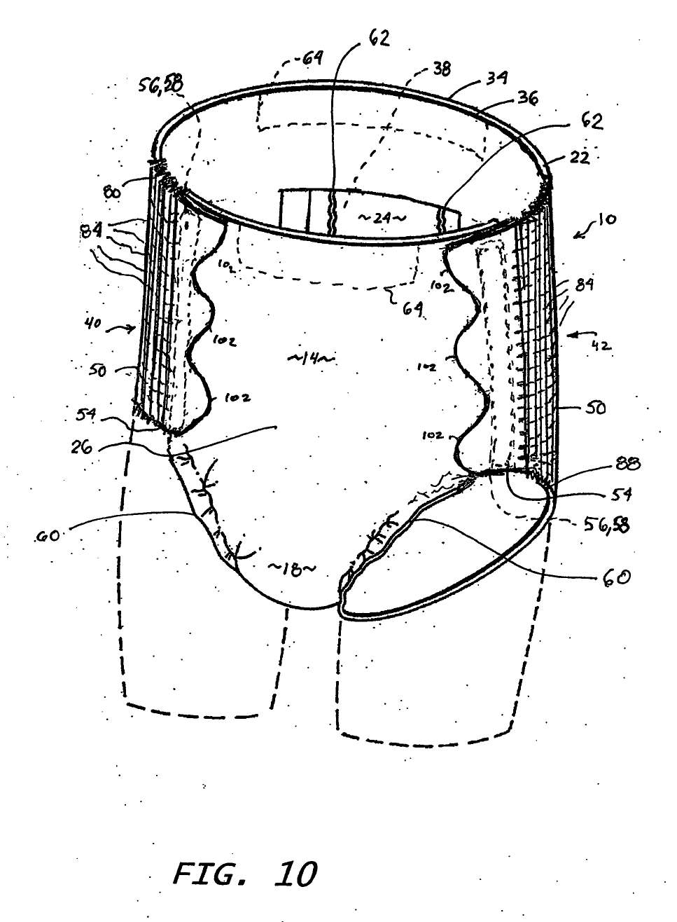

FIG. 10 illustrates yet another embodiment of the present invention wherein side panels 40, 42 each comprise a finger-lift structure. In the embodiment of FIG. 10, the finger-lift structure comprises three tabs 102. A diaper 10 with two tabs 102 or more than three tabs 102 may also be practicable. Tabs 102 may assume a variety of different shapes and sizes. Tabs 102 are preferably extensions of the material defining inextensible zone 54. When side panels 40, 42 are properly secured to the front waist portion 14 (as illustrated in FIG. 10), tabs 102 do not engage hook element 58 of the refastenable fastener. As a result, tabs 102 define unsecured free ends of the side panels 40, 42 which facilitate the release of the refastenable fastener 56, 58 during inspection or removal of the diaper 10. Similar tab-containing configurations are equally applicable to the side panel constructions of FIGS. 2, 4 and 8.

A variety of different manufacturing methods may be utilized to form the improved diaper 10 of the present invention. One method of manufacturing diaper 10 may include the step of forming the pair of side panels by joining the two plies of material 80, 82 together while incorporating the elastic ribbons 84 within the elastic zone 50. The side panels 40, 42 can then be joined along attachment lines 90 to the absorbent chassis 12 at opposed side edges thereof so that the side panels 40, 42 extend outwardly from said side edges of said absorbent chassis 12. The fastener components 58 can be joined to the front waist portion 14 in a generally longitudinal direction. Fastener component 56 which is within extensible zones 54 of the side panels 40,42 may be simply defined by the material surface 82. One of ordinary skill the in art would appreciate additional useful manufacturing methods or steps.

The diaper 10 may be fitted to the wearer in a pull-on, or pant-like configuration. The diaper 10 may be constructed generally as stated above to form a pant-like article having a waist hoop and a pair of leg openings. In this configuration, the wearer's legs are directed through the leg openings. Once the wearer's legs are through the leg openings, the diaperer can then pull the waist hoop of the diaper up over the wearer's hips such that the waist hoop encircles the waist of the wearer.

Alternatively, the diaper 10 may be applied to the wearer in a conventional configuration. To do so, the rear waist region 16 is preferably placed under the wearer's back. The remainder of the diaper 10 is preferably then drawn between the wearer's legs such that the other waist region, preferably the front waist region 14, is positioned across the front of the wearer. The diaperer preferably then grasps the side panels 40, 42 and wraps them around the waist of the wearer. Using the diaper embodiment of FIG. 1, the diaperer then engages the fastener component 56 disposed on the inner surface of the side panel 40, 42 and the fastener component 58 disposed on the garment facing surface 26 in the front waist region 14. This forms a waist closure on each side of the wearer and completes the construction of the diaper in a conventional configuration. Alternatively, when using the diaper embodiment of FIG. 2 the diaperer engages the fastener components 56, 58 disposed on side panel margins 70, 72 to define the pant-like article.

Diaper 10 of the present invention may be fitted to the wearer in any other suitable manner, including a combination of the steps described above with regard to the conventional and pull on embodiments. In any configuration, the fastener system of the present invention provides the user with easy access to inspect the diaper 10 for soiling. Further, the fastening system gives the user the option as to how the diaper 10 will be removed. The diaper 10 can be removed by pulling the diaper 10 down and off the wearer without disengaging the elements of the fastening system, or by disengaging at least one of the engaging components from its corresponding landing component and removing the diaper 10 from around the wearer's waist.

The present invention advantageously provides a pant-like, refastenable disposable absorbent article. The side panels are releasably prefastened to allow the absorbent article to be pulled up or down over the hips of the wearer such as conventional training pants. Moreover, the fastener system can be used to refastenably engage and adjust the front and back waist portions of the absorbent article to maintain the absorbent article about the waist of the wearer after the article has been pulled on in a similar manner to conventional diapers.

As a result, the absorbent articles of the present invention are designed to conform to the body of the wearer to effectively contain bodily exudates while still being capable of being reliably pulled up and down over the hips of the wearer to assist in the toilet training of the wearer. Moreover, similar to conventional diapers, the absorbent articles of the present invention can advantageously be applied to and removed from the wearer with relative ease and cleanliness.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A disposable absorbent article comprising:
an absorbent chassis which defines a front waist region, a rear waist region, a crotch region which extends between and connects said waist regions, a pair of longitudinally opposed end edges, and a pair of laterally opposed side edges; and
at least one laterally extensible side panel defining a pair of inextensible zones and an intermediate elastic zone disposed between the pair of inextensible zones, said side panel being permanently attached to the absorbent chassis at one of the pair of inextensible zones and being refastenably attached to the absorbent chassis at the other of the pair of inextensible zones via a first refastenable fastener component.

2. The absorbent article of claim 1 wherein the side panel is permanently attached at the rear waist region and refastenably attached to the absorbent chassis at the front waist region.

3. The absorbent article of claim 2 wherein the side panel is refastenably attached upon an outer surface of absorbent chassis.

4. The absorbent article of claim 2 wherein the side panel is refastenably attached upon an inner surface of absorbent chassis.

5. The absorbent article of claim 1 wherein the side panel is permanently attached at the front waist region and refastenably attached to the absorbent chassis at the rear waist region.

6. The absorbent article of claim 5 wherein the side panel is refastenably attached upon an outer surface of absorbent chassis.

7. The absorbent article of claim 5 wherein the side panel is refastenably attached upon an inner surface of absorbent chassis.

8. The absorbent article of claim 1 wherein the first fastener component is part of a refastenable system including a second fastener component disposed upon the absorbent chassis, and wherein the first and second fastener components cooperate to attach the side panel to the absorbent chassis.

9. The absorbent article of claim 8 wherein the second fastener component is disposed within the front waist region of the absorbent chassis.

10. The absorbent article of claim 8 wherein the second fastener component is disposed within the rear waist region of the absorbent chassis.

11. The absorbent article of claim 8 wherein the first fastener component and the second fastener component are components of a refastenable hook and loop-type fastener.

12. The absorbent article of claim 1 wherein the first fastener component is a loop-type fastener.

13. The absorbent article of claim 8 wherein the loop-type fastener is defined by a fiber surface of the side panel.

14. The absorbent article of claim 8 wherein the loop-type fastener is defined by a fiber surface of the absorbent chassis.

15. The absorbent article of claim 1 wherein the first fastener component is a hook-type fastener disposed entirely within an associated inextensible zone.

16. The absorbent article of claim 1 wherein the elastic zone of the side panel extends substantially between a waist edge and a leg opening edge of the side panel.

17. The absorbent article of claim 1 wherein at least one of inextensible zones extends substantially between a waist edge and a leg opening edge of the side panel.

18. The absorbent article of claim 1 wherein the first fastener component extends generally between a waist edge and a leg opening edge of the side panel.

19. The absorbent article of claim 1 wherein the first fastener component is elongated in a longitudinal direction of the diaper.

20. The absorbent article of claim 1 wherein the elastic zone and the pair of inextensible zones are comprised of generally inextensible materials.

21. The absorbent article of claim 1 wherein the side panel includes one or more single ply elements.

22. The absorbent article of claim 1 wherein the side panel comprises a top ply of material and a bottom ply of material which together define portions of the elastic zone and the pair of inextensible zones.

23. The absorbent article of claim 17 wherein the elastic zone further comprises an elastic member joined to the top and bottom plies of material.

24. The absorbent article of claim 1 wherein the elastic member comprises a plurality of elastic ribbons which extend along at least a portion of the side panel generally between the pair of inextensible zones.

25. The absorbent article of claim 24 wherein an elastic ribbon is intermittently secured to an element of the side panel.

26. The absorbent article of claim 1 wherein each side panel comprises a pair of separable, refastenable side margins.

27. The absorbent article of claim 26 wherein one of the pair of side margins is joined to the absorbent chassis at the front waist region and the other side margin is joined at the rear waist region.

28. The absorbent article of claim 27 wherein each of the side margins defines a pair of inextensible zones.

29. The absorbent article of claim 28 wherein a refastenable fastener system is defined by a pair of fastener components provided upon each side panel.

30. The absorbent article of claim 29 wherein each of the pair of fastener components is disposed within an inextensible zone of an associated side margin.

31. The absorbent article of claim 1 wherein the side panel defines m elastic zones and a m+1 inextensible zones, where m is greater than 2.

32. A disposable absorbent article comprising:
an absorbent chassis which defines a front waist region, a rear waist region, a crotch region which extends between and connects said waist regions, and a pair of laterally opposed side edges; and
at least one laterally extensible side panel defined at least in part by a pair of inextensible zones and an intermediate elastic zone defined between the pair of inextensible zones, said side panel being connected to the absorbent chassis at an end portion and being temporarily attached to the absorbent chassis at a free end portion via a refastenable fastener component, wherein a portion of each of the elastic zone and inextensible zones defines a leg opening edge of the absorbent article.

33. The disposable absorbent article of claim 32 wherein the leg opening edge and one of the pair of side edges defines a perimeter of a leg opening when the free end portion of the side panel is secured to the absorbent chassis.

34. The disposable absorbent article of claim 32 wherein the leg opening edge defines portions which are linearly shaped or portions which are non-linearly shaped or both.

35. The disposable absorbent article of claim 32 wherein the elastic zone includes an elastic element joined to a ply structure in a pretensioned state.

36. The disposable absorbent article of claim 35 wherein the elastic element is intermittently joined to the ply structure.

37. The disposable absorbent article of claim 35 wherein the elastic element comprises a plurality of elastic ribbons.

38. The disposable absorbent article of claim 37 wherein the plurality of elastic ribbons extend along at least a portion of the side panel.

39. The disposable absorbent article of claim 32 wherein portions of each of the elastic zone and inextensible zones define part of a waist edge.

40. The disposable absorbent article of claim 32 wherein the elastic zone extends generally between a waist edge and the leg opening edge.

41. The disposable absorbent article of claim 32 wherein at least one inextensible zone extends generally between a waist edge and the leg opening edge.

42. The disposable absorbent article of claim 32 wherein the side panel defines a pair of elastic zones and a third inextensible zone between the pair of elastic zones.

43. The disposable absorbent article of claim 32 wherein at least one of the pair of inextensible zones is defined by a single ply of material.

44. The disposable absorbent article of claim 32 wherein the refastenable fastener component extends generally between a waist edge of the absorbent article and the leg opening edge.

45. The disposable absorbent article of claim 32 wherein the refastenable fastener component is disposed within the inextensible zone nearest to the free end portion.

46. The disposable absorbent article of claim 32 wherein the refastenable fastener component is elongated and aligned generally in a longitudinal direction of the absorbent article.

47. The disposable absorbent article of claim 32 wherein the side panel is temporarily attached at the front waist region.

48. The disposable absorbent article of claim 32 wherein the side panel is temporarily attached at the rear waist region.

49. A pant-like, refastenable, disposable absorbent article which defines a front waist region, a back waist region, a crotch region which extends between and connects said waist regions, a longitudinal direction and a lateral direction, said absorbent article comprising:
an absorbent chassis which defines an exterior surface, an interior surface opposite said exterior surface, a pair of laterally opposed side edges;
a pair of opposed side panels which extend outwardly from said side edges of said absorbent chassis, wherein each of said opposed side panels defines a first inextensible panel which is permanently attached proximate to said side edge of said absorbent chassis and an elastic panel which is permanently attached to the first inextensible panel and a second inextensible panel which is permanently attached to the elastic panel; and
a fastener system includes first fastener components defined upon the absorbent article and second fastener components defined within the second inextensible panels, said fastener system refastenably securing the side panels to the absorbent chassis to define a pant-like absorbent article having a pair of leg openings, and wherein each of the first and second inextensible panels and the elastic panel define a portion of a leg opening.

50. The absorbent article of claim 49 wherein the side panel is a ply structure of substantially inextensible material.

51. The absorbent article of claim 50 wherein the side panel includes an elastic element which is joined to portions of the ply structure in a pretensioned state.

52. The absorbent article of claim 49 wherein the side panels define a portion of a waist edge of the absorbent article.

53. The absorbent article of claim 52 wherein the inextensible panels extend generally between the waist edge and an edge of the leg opening.

54. The absorbent article of claim 49 wherein the second fastener component of the side panel is elongated and extends generally between a waist edge and a leg opening edge.

55. The absorbent article of claim 49 wherein portions of the edges of the leg openings include are non-linearly configured.

56. The absorbent article of claim 49 wherein each side panel further defines a pair of elastic panels and a third inextensible panel between the pair of elastic panels.

57. The absorbent article of claim 49 wherein the side panels extend from the back waist region of the absorbent article.

58. The absorbent article of claim 49 wherein the side panels extend from the front waist region of the absorbent article.

59. A disposable absorbent article comprising:
an absorbent chassis which defines a front waist region, a rear waist region, a crotch region which extends between and connects said waist regions, and a pair of laterally opposed side edges; and
at least one laterally extensible side panel defined at least in part by a pair of side margins, wherein each side margin includes a pair of inextensible zones and an intermediate elastic zone defined between the pair of inextensible zones, said side margins being connected to the absorbent chassis at ends proximate to the pair of side edges and being refastenably attached together at second free ends via refastenable fastener components, wherein a portion of each of the elastic zone and inextensible zones define a leg opening edge of the absorbent article.

60. The disposable absorbent article of claim 59 wherein the leg opening edges and portions of the pair of side edges define leg openings when the free ends of the side margins are secured together.

61. The disposable absorbent article of claim 59 wherein the side margins are each defined as a ply structure of substantially inextensible material.

62. The disposable absorbent article of claim 61 wherein the elastic zones include elastic elements joined to portions of the ply structure in a pretensioned state.

63. The disposable absorbent article of claim 62 wherein the elastic elements comprise a plurality of elastic ribbons.

64. The disposable absorbent article of claim 63 wherein the plurality of elastic ribbons extend laterally relative to the absorbent chassis.

65. The disposable absorbent article of claim 59 wherein a waist edge is defined by a portion of the elastic zone.

66. The disposable absorbent article of claim 65 wherein the elastic zone extends generally between the waist edge and the leg opening edge.

67. The disposable absorbent article of claim 65 wherein at least one inextensible zone extends generally between the waist edge and the leg opening edge.

68. The disposable absorbent article of claim 65 wherein the inelastic zones extend generally between a waist edge and the leg opening edge.

69. The disposable absorbent article of claim 65 wherein the leg opening edge is generally non-linearly configured.

70. The disposable absorbent article of claim 65 wherein the refastenable fastener components are substantially disposed within the inextensible zones.

71. The disposable absorbent article of claim 65 wherein at least one of the refastenable fastener components extends generally between a waist edge of the absorbent article and the leg opening edge.

72. The disposable absorbent article of claim 71 wherein the refastenable fastener components are elongated and aligned generally in a longitudinal direction of the absorbent article.

73. A packaged assembly of prefastened disposable absorbent articles comprising:
a plurality of absorbent articles each having an absorbent chassis which defines a front waist region, a rear waist region, a crotch region which extends between and connects said waist regions, a pair of longitudinally opposed end edges, and a pair of laterally opposed side edges, and at least one laterally extensible side panel defining a pair of inextensible zones and an intermediate elastic zone disposed between the pair of inextensible zones, said side panel being permanently attached to the absorbent chassis at one of the pair of inextensible zones and being refastenable attached to the absorbent chassis at the other of the pair of inextensible zones via a refastenable fastener component.

74. The packaged assembly of claim 73 wherein the absorbent articles each include a pair of side panels.

75. The packaged assembly of claim 73 wherein the pair of inextensible zones and elastic zone are defined by a top layer of material and a bottom layer of material.

76. The packaged assembly of claim 75 wherein at least one of the top layer or bottom layer of material is a generally nonextensible material.

77. The packaged assembly of claim 76 wherein the elastic zone includes an elastic element connected in a pretensioned state to the nonextensible material.

78. The packaged assembly of claim 77 wherein the pair of inextensible zones and elastic zone defines portions of a leg opening edge.

79. The packaged assembly of claim 73 wherein the side panel defines a pair of elastic zones and a third inextensible zone between the pair of elastic zones.

80. A pant-like disposable absorbent article manufactured by a method comprising the steps of:
providing an absorbent chassis having an exterior surface, an interior surface opposite said exterior surface, and a pair of laterally opposed side edges;
joining a pair of side panels to the absorbent chassis at opposed side edges thereof so that the side panels extend outwardly from said side edges of said absorbent chassis, wherein each side panels defines an elastic panel interconnected between a pair of generally non-stretchable panels, wherein one of the pair of non-stretchable panels is permanently joined to the absorbent chassis and the other one of the pair of non-stretchable panels is provided with a refastenable fastener component; and
joining the fastener components of the pair of side panels with complementary fastener components provided upon the absorbent chassis to define a pant-like absorbent article having a pair of leg openings, and wherein the pair of non-stretchable panels and the elastic panel each define portions of a pair of the leg openings.

81. The method of claim 80 wherein the step of joining the pair of side panels includes a step of adhesive bonding material of the side panel to the absorbent chassis.

82. The method of claim 80 wherein the step of joining the pair of side panels includes a step of aligning a waist edge of the side panel with a waist edge of the absorbent chassis.

83. The method of claim 80 wherein the step of joining the pair of side panels includes a step of joining two pairs of side margins to the absorbent chassis.

84. The method of claim 80 wherein the step of joining the pair of side panels results in the pair of side panels being joined at a rear waist region of the absorbent article.

85. The method of claim 80 wherein the step of joining the pair of side panels results in the pair of side panels being joined at a front waist region of the absorbent article.
